(19) United States
(12) Patent Application Publication
(10) Pub. No.: US 2003/0228632 A1
(43) Pub. Date: Dec. 11, 2003

(54) Stable RXR expressing cell line

(75) Inventors: Jyotirmoy Kusari, Irvine, CA (US); Sheila X. Zhou, Irvine, CA (US); Hongzhi Liu, Irvine, CA (US); Ramilla O. Lewis, Lake Forest, CA (US); Roshantha A. Chandraratna, Laguna Hills, CA (US)

Correspondence Address: Carlos A. Fisher, ALLERGAN, INC., T2-7H, 2525 Dupont Drive, Irvine, CA, 92612, US

(73) Assignee: Allergan Sales, Inc.

(21) Appl. No.: 09/850,835

(22) Filed: May 8, 2001

Related U.S. Application Data
(60) Provisional application No. 60/203,116, filed on May 8, 2000.

(51) Int. Cl. G01N 33/53; G01N 33/567; C07K 14/705

(52) U.S. Cl. 435/7.2; 435/325; 530/350

(57) ABSTRACT

Stable cell lines which express retinoid receptors and the insulin receptor are prepared and are useful in identifying agonists and antagonists of retinoid receptors. Agonists and antagonists of the RXR receptor can be determined using the cell lines of the invention which are producers of RXR alone; agonists and antagonists of other retinoid receptors can be determined using cell lines transfected with RXR and the desired retinoid receptor.

### TECHNICAL FIELD

The invention relates to a cell line that stably expresses the retinoid receptor RXR and is thus useful for screening ligands that bind RXR and elicit biological responses such as inhibition of cell growth. The cell line is also useful as a host for co-expression with constructs encoding receptors that form heterodimers with RXR.

### BACKGROUND ART

The retinoid receptor, RXR, is of particular importance because it is able both to function as a homodimer and also to form heterodimers with other receptors in the family of nuclear receptors to which it belongs. RXR homodimerization in vitro is induced by 9-cis retinoic acid. See Zhang, X, et al., Nature (1992) 3508:587-591. It is also known that RXRs can form heterodimers with Vitamin D receptor (VDR), thyroid hormone receptor (TR), peroxisome-proliferator activated receptor (PPAR), COUP and ARP-1, as well as retinoic acid receptor (RAR). See Nagpal, S., et al., Curr. Pharmaceut. Des. (1996) 2:295-316.

These receptors modulate transcription by either inhibiting or enhancing transcription by direct interaction of the ligand-bound receptors with nuclear DNA. Thus, in order to exert their physiological function, the receptors inside the nucleus or cytoplasm of the target cell first bind to ligand (which ligand must itself penetrate the cell membrane), presumably undergo a conformational change, and are then able to interact with appropriate portions of the genome to regulate transcription. These receptors participate in a complex and important scheme for regulating cell growth and differentiation. Considerable detail about the nature of the interaction of the ligand-bound receptors to the gene has been described. See, for example, Mangelsdorf, D.J., et al., in “The Retinoids: Biology, Chemistry and Medicine” 2nd Ed.; Spom, M.B., et al, Eds., Raven Press New York (1994) pages 319-349.

In order to provide a convenient system for analysis of this regulatory pathway, and for screening ligands that bind to the receptors which participate in it, it would be advantageous to have a cell line which is stably transformed with these receptors, especially where the cell line also stably expresses the insulin receptor (IR). This is especially important because in general, the members of the steroid nuclear receptor superfamily, and in particular, the peroxisome-proliferator activated receptor (PPAR) are known to affect the insulin response. Further, it is known that the insulin receptor has effects on transcription of some receptors and affects phosphorylation of proteins that participate in various cell signaling functions. No such stably transformed cell line currently exists, to applicants' knowledge. This is because, typically, overexpression of the retinoid receptors causes differentiation of the cell lines where expression occurs, and their immortality is lost. For example, 3T3 cells modified to contain an expression system for RXR differentiate into adipocytes.

It has now been found possible to obtain a stable cell line comprising the retinoid receptors and their family members that participate in this regulatory scheme, said cell line also comprising the insulin receptor.

### DISCLOSURE OF THE INVENTION

The invention provides a stable cell line which expresses members of the superfamily of ligand-dependent transcription factors that includes receptors for steroid and thyroid hormones, Vitamins A and D and retinoic acid, as well as expressing the insulin receptor. The cell line is obtained by transfecting the HircB cell line or other IR expressing cell line with the appropriate constructs containing the genes encoding the desired receptors under control of mammalian cell-compatible promoters, such as viral promoters. The transfection efficiency is high and the receptors may be overexpressed without causing differentiation of these cells.

Thus, in one aspect, the invention is directed to a stable cell line expressing a retinoid receptor in amounts sufficient to display biological activity when treated with a ligand for said receptor, as well as expressing the insulin receptor. The cell lines are convenient tools for identifying agonists and antagonists of the relevant retinoid receptor and thus, in other aspects, the invention is directed to methods to screen for agonists and antagonists using the cell lines of the invention. The physiological relevance of these agonists and antagonists is improved by virtue of the inclusion of the insulin receptor in these cell lines.

### BRIEF DESCRIPTION OF THE DRAWINGS

FIG. 1 shows the nucleotide sequence (FIG. 1A) and deduced amino acid sequence (FIG. 1B) for human insulin receptor.

FIG. 2 shows a diagram of the pFLAG-CMV-2 expression vector useful in the invention.

FIG. 3 shows the nucleotide sequence (FIG. 3A) and the deduced amino acid sequence (FIG. 3B) for the human gene encoding human RXRA.

FIG. 4 is a photocopy of a Western blot showing the expression of RXRα in HircB clones 1, 2 and 3 prepared according to the invention. Lane 1 is a homogenate from HircB cells, lanes 2-4 are homogenates from clones 1-3 respectively.

FIG. 5 shows cell counts obtained after four days when the cells of the invention were treated with an RXR agonist as compared to the effect on the parental cell line.

FIG. 6 shows a dose response correlation for the effect of an RXR agonist on cell growth in the cell line of the invention.

FIG. 7 shows the effect of various RXR ligands on cell growth in the cell line of the invention.

FIG. 8 is a photocopy of a Western blot which shows the effect of the presence of RXRα in the cell line on the ability of insulin to maintain MEK in the phosphorylated state.

FIG. 9 is a photocopy of a Western blot which shows the effect of the presence of RXRα in the cell line on the ability of insulin to maintain MAP-kinase in the phosphorylated state.

### MODES OF CARRYING OUT THE INVENTION

The “retinoid receptors” that are produced by the stable cell line of the invention include receptors that belong to the superfamily of transcription factors that are activated by ligands which enter the cell by virtue of their ability to permeate the cell membrane. Prominent among these receptors are those which respond to trans or 9-cis retinoic acid—RAR and RXR, respectively. Human RAR and RXR receptors each exhibit three known isoforms, RARα, RARβ, RARγ and RXRα, RXRβ, RXRγ. RAR and RXR receptors differ in the nature of the ligands with which they associate and by the nature of agonists and antagonists with respect to their activity. Also included among “retinoid receptors” as defined herein are the related receptors for Vitamin D, steroids, thyroid hormone receptor, peroxisome-proliferator activated receptor (PPAR) and the orphan receptors, COUP, ARP-1, and the like.

Throughout the specification and in the claims, the term “express” or “expression” is used to describe the transcription and translation of a nucleotide sequence into protein. Applicants understand that technically “expression” refers to this sequence of events and thus it is correct to refer to the “expression” of a nucleotide sequence, the end product of which will be a protein. However, common usage permits the use of this term to refer in an abbreviated way to the end result so that “expression” of the end product protein is sometimes referred to. It will be clear, by virtue of this explanation, that “expression” refers to the process and “expressed” will be used to refer both to the nucleotide sequence that is transcribed and translated and to the protein that is produced.

The invention employs standard recombinant techniques to insert the expression system for the desired retinoid receptor into an appropriate expression vector and for modifying the host cell line. The stability of the resulting cell line is apparently a function of the nature of the host. The preferred host cell in this instance is an established cell line, long well-known in the art, designated HircB. HircB is a Rat 1 fibroblast cell line which expresses the human insulin receptor. It can be propagated in DMEM-F12 containing 10% fetal calf serum, 50 μg/ml gentamycin, 2 mM glutamax and 500 nM methotrexate. The preparation of this cell line is described in detail in McClain, D. A., et al., J. Biol. Chem. (1987) 14663-14671. The description of the construction of the expression vector used to modify Rat 1 cell lines and the procedure for transforming and maintaining the Rat 1 cell line as the host cell herein, HircB, on page 14664 is incorporated herein by reference.

Briefly, an expression plasmid pCVSV-HIRc was prepared which contains the origin of replication and the ampicillin-resistance gene of E. coli plasmid pBR322, DNA-encoding mouse dihydrofolate reductase under control of the SV40 early promoter, and a 2279-5231 base pair XbaI/DraI fragment comprising the complete human insulin receptor (HIR) coding sequence under the SV40 early promoter control. (The parent plasmid into which the HIR coding sequence was inserted is described in European publication No. 117060.) The prepared construct has the pre-insulin receptor initiation codon as well as a 50 nucleotide sequence from the 5′ untranslated region and a 10 basepair polylinker region of pUC12 joined to the SV40 early promoter. HIR 3′ untranslated sequences continue 1018 nucleotides downstream from the stop codon.

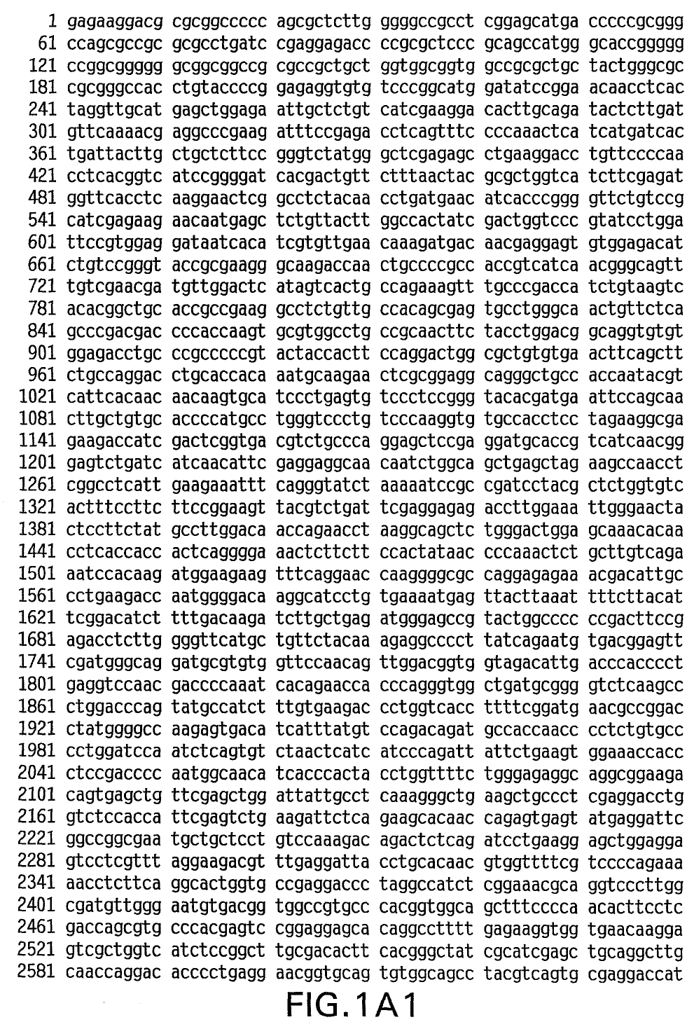

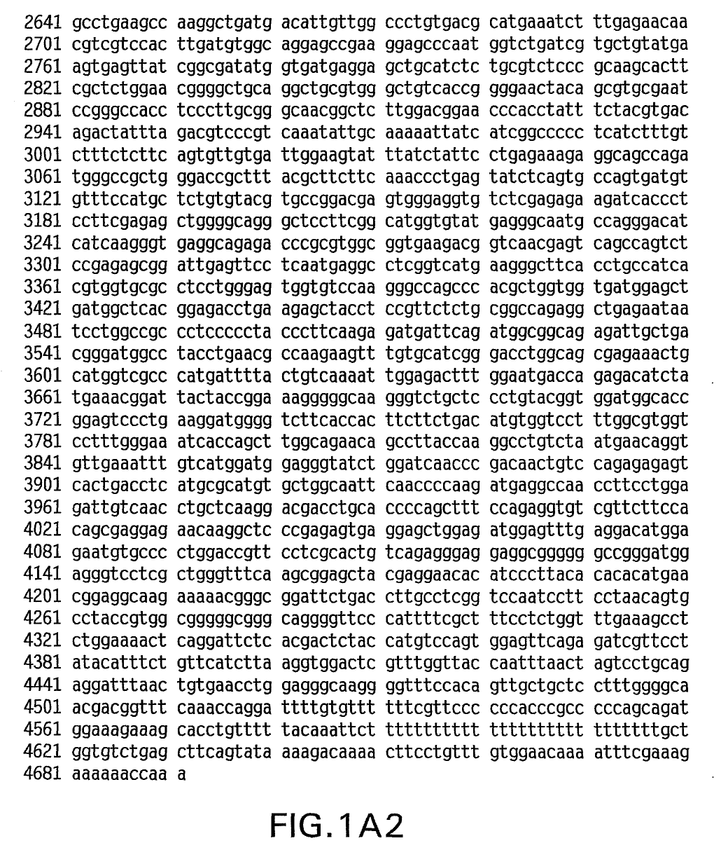

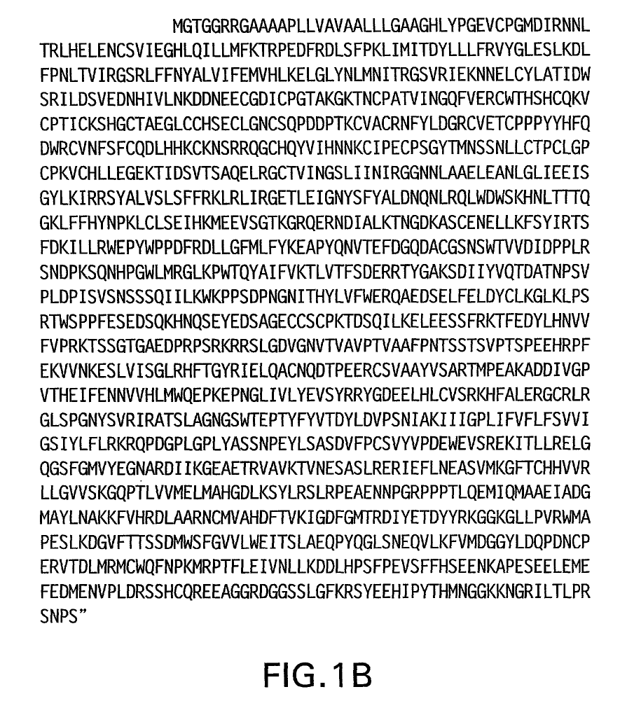

The HIR expression plasmid, designated pCVSV-HIRc, was amplified in E. coli. Purified rat embryo fibroblasts (Rat 1) cells (10⁶ cell/dish) were co-transfected with this plasmid and pSV-Neo (500 ng) using calcium phosphate precipitation with glycerol shock after 4 hours exposure to the precipitate. After 48 hours exposure to the plasmid, the cells were trypsinized, re-plated at lower density (1:4) and selected with 400 μg/ml G418 for 2 weeks. Independent colonies were picked, cultured and amplified stepwise using methotrexate at various concentrations and resistant cell lines expressing high levels of human insulin receptor were screened by binding radiolabeled insulin in the presence and absence of unlabeled insulin. (The original Rat 1 cells were grown in F12/Dulbecco's modified Eagles medium (50:50) supplanted with 10% fetal bovine serum. For methotrexate selection, cells were grown in the same medium containing 7% extensively dialyzed FBS and cultures were maintained in a humidified atmosphere at 5% CO₂ at 37° C.) One of the cell lines isolated was designated HircB. (For convenience, FIG. 1 shows the nucleotide sequence encoding, and the deduced amino acid sequence for, human insulin receptor.)

It will be apparent to the skilled artisan that by following similar procedures, additional cell lines expressing the insulin receptor are readily obtained. These cell lines, too, are suitable for preparing the cell lines of the invention. Alternatively, cell lines modified as described herein to express at least one retinoid receptor can be subsequently modified to express the insulin receptor.

By way of illustration, transfection of the HircB cell line with an appropriate retinoid expression vector is accomplished typically by co-transformation with a selective marker construct and the transformed cell lines are then maintained in the presence of selection medium. Production of the desired retinoid receptor can then be confirmed by a variety of methods including Western blot, precipitation with anti-retinoid receptor antibody, and the like. In a preferred embodiment, the retinoid receptor is an RXR receptor.

The stably transformed cell lines are useful in screening for factors that agonize and antagonize the relevant receptor. Cells transfected with one of the isoforms of RXR, or with different isoforms of RXR, are responsive to certain ligands since homodimers of RXR are themselves biologically active. Alternatively, the cells can be co-transfected with another member of the retinoid family which forms a heterodimer with RXR and the resulting cell line used as a screening tool for ligands relevant to these receptors. Thus, further transfection with similar constructs containing, for example, RAR, or PPAR in one or more isoforms can also be produced. Particularly preferred are cells which not only express the IR, but are cotransfected so as to express both RXR receptors and RAR receptors, RXR receptors and Vitamin D receptor, RXR receptor and steroid receptors, RXR and thyroid hormone receptors, and RXR and PPAR. Particularly preferred is the combination of RXR and PPAR, especially RXR and PPARγ.

In one preferred embodiment, the cell line of the invention expresses the RXR receptor, preferably the RXRα isoform. Cell growth can be studied in such a cell line by investigating the effects of ligands for the RXR receptor itself on cell growth. The known ligand for the RXR receptor is 9-cis retinoic acid. However, other agonists and antagonists can be found. Thus, in one aspect, the invention is directed to identifying an agonist for the RXR receptor which method comprises contacting the cells of the invention which express the RXR receptor with a candidate molecule and determining the effect of this molecule on cell growth. Cell growth—i.e., proliferation—can be measured by a variety of techniques, such as measurement of the uptake of tritiated thymidine, incorporation of BrdU and the MTT assay. The pathways mediated by the receptors in general are complex and the resultant may either be diminution or enhancement of growth. Thus, a change in growth patterns in either direction is indicative of an agonist.

Antagonists can be identified by simultaneously treating cells expressing the RXR receptor with a known agonist and a candidate antagonist. An diminution in the effectiveness of the agonist indicates antagonist activity.

As noted above, cells transfected with RXR receptors are preferred because in order to exert their transcription regulation effect, these receptors can function as homodimers. However, the use of the cell lines can also be expanded by co-transfection with a receptor, such as Vitamin D, steroid receptor, thyroid receptor, PPAR, RAR and the like, with which RXR forms heterodimers, in order to evaluate the activity of the companion receptor in the presence of various test compounds. Thus, the heterodimer of RAR and RXR is activated by an RAR ligand; the heterodimer of PPAR and RXR is activated by a PPAR ligand. Of course, the order of transformation does not matter so that stable cell lines which contain, for example, only RAR or PPAR or VDR receptors are included within the invention as well. Screening for antagonists and agonists for such cells is analogous to the methods described above; typically, where a heterodimer is formed with RXR, the agonist or antagonist is characteristic of the receptor which is heterologous to RXR.

Where the IR-expressing cells have been modified to express both RXR and an additional retinoid receptor, it is preferred that both retinoid receptors remain stably expressed in the cell line. However, also included within the invention are cell lines wherein only the RXR receptor is stably expressed and the additional retinoid receptor is produced only transiently. Thus, cells which stably express IR and RXR may transiently express, in addition, RAR, VDR, PPAR and the like.

Thus, by selecting cells that produce the IR, for example, HircB cells as a host, a repertoire of retinoid receptor expressing cells can be obtained, which are stable and which have the additional feature of IR interaction. These cells are useful tools in identifying ligands for the receptors that modulate transcription and cell growth. The cell lines are also useful to explore the mechanism whereby this occurs, including the identification of the hormone receptor sites on the genome that respond to the activated receptors.

The following examples are intended to illustrate but not to limit the invention. They describe the production of a particular retinoid expression cell line, designated herein HircRXRα. However, similar constructs to those of Example 1 that include other retinoid receptors whose coding sequences are available in the art and using other cell lines expressing the IR can be prepared and, by employing the techniques of cell line transformation described in Example 2, a variety of retinoid receptor expressing cell lines can be obtained. Each of these cell lines is useful in investigating the effects of particular ligands or in cell growth and elucidating the mechanisms whereby such effects occur.

### EXAMPLE 1

### Preparation of an Expression Vector for RXRα

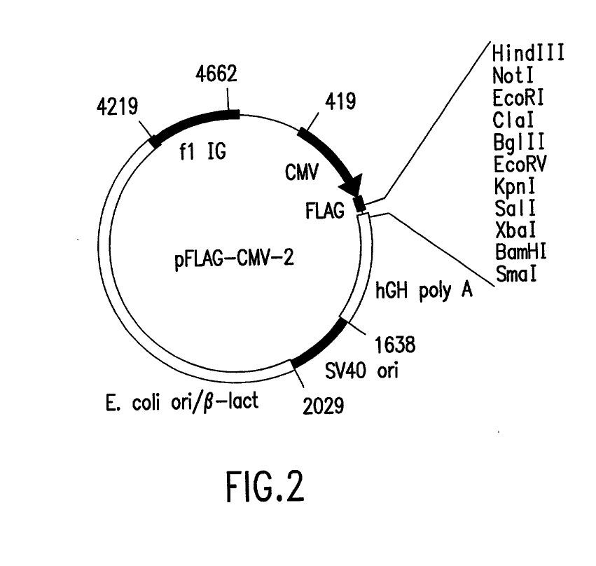

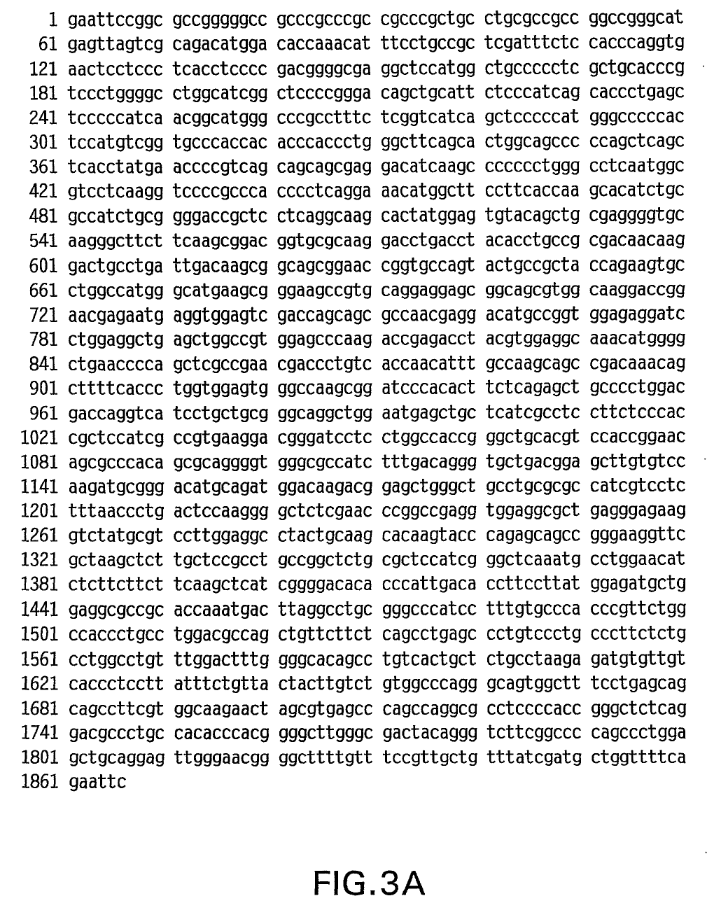

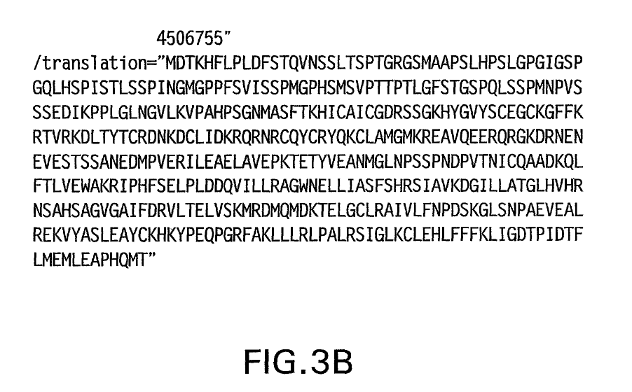

The expression vector pFLAG-CMV-2, diagramed in FIG. 2, and available from Sigma Chemical Co. (St. Louis, Mo.) was used as the host vector. The 1.5 kb cDNA clone of human RXRα, the nucleotide sequence of which is shown in FIG. 3 as NCBI accession No. NM002957, was introduced as a KpnI/EcoRI fragment into the host vector. The resulting construct contains the RXRα coding sequence under control of the CMV promoter and terminated by hGH polyA.

### Example 2

### Preparation of the Hirc-RXRα Cell Line

The expression plasmid for RXRα as prepared in Example 1 was co-transfected into HircB cells along with pSV-HY890 in the presence of lipofectamine using hygromycin (400 μg/ml) for selection. Derivative cell lines were established by clonal propagation of cells expressing high levels of RXRα protein. These cells were maintained in the presence of 400 μg/ml hygromycin.

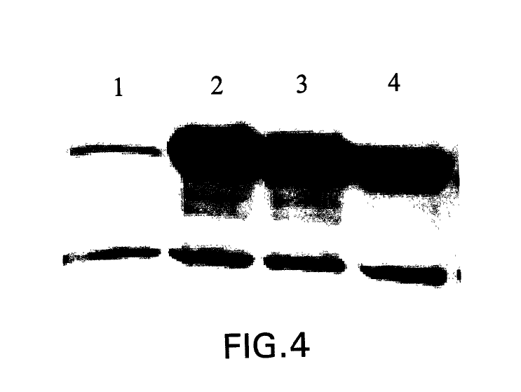

Three clones expressing high levels of RXR were identified by Western blot as follows: The cells were grown to near confluence and then solubilized in Laemmli's sample buffer. The cell homogenates were segregated by SDS-PAGE and immunoblotted with anti-RXRα antibodies. As shown in FIG. 4, parental HircB cells (lane 1) show little expression of RXRα, but clones 1, 2 and 3, shown in lanes 2, 3 and 4 respectively show high expression of RXRα.

### Example 3

### Effect of Agonists and Antagonists for RXR Receptors

Cell lines, designated HircRXRα, obtained as described in Example 2 were then treated with known agonists and antagonists for the RXR receptor. These are shown in Table 1.

TABLE 1

| RXR Agonists | Antagonists |
|---|---|
| AGN 4204 | AGN 5393 |
| AGN 5184 | |
| AGN 5203 | |

The effect on cell growth was evaluated by plating either a HircB cells (as a control) or HircRXRα cells in 6 well plates with 0.5×10⁵ cells per well in 10% FBS/DMEM/F12. The next day, the medium was replaced with a specified concentration of agonist, antagonist or both or with 0.1% DMSO in 10% FBS/DMEM/F12. Analyses were performed in triplicate.

After incubation with agonist for 2 days, the change in cell number and morphology in the various HircRXRα cell samples were observed under the microscope. Alternatively, cell growth was measured using the MTT assay using absorbance at 590 nm. Mosmann, T., et al., J Immunol. Meth. (1983) 65:55-62.

Based on direct microscopic observation, cells treated with as little with 1 nM AGN 4204 showed greatly reduced growth after two days; the reduction in growth was maintained after four days. However, when the antagonist AGN 5393 was added at 10 nM concentration, after four days of treatment with AGN 4204, in the presence of this concentration of AGN 5393, no inhibition of growth was observed. Concentrations of AGN 5393 at 1 nM did not appear to be effective. (Data not shown)

Based on direct microscopic observation of cell growth, the agonist AGN 5184 at 1 nM concentration decreased the cell numbers after both two and four days. AGN 5203 at 1 nM concentration appeared to decrease growth after two days but growth was restored almost to normal levels after four days. (Data not shown)

FIG. 5 shows a comparison of the effect of the RXRα agonist AGN 4204 on growth of HircRXRα cells as compared to HircB cells. As seen in FIG. 5, when cell counts were performed after four days of treatment with 1 nM AGN 4204, growth in the HircRXRα cells was reduced to approximately one-third of that observed when no agonist was added; there was no demonstrable effect on HircB cells.

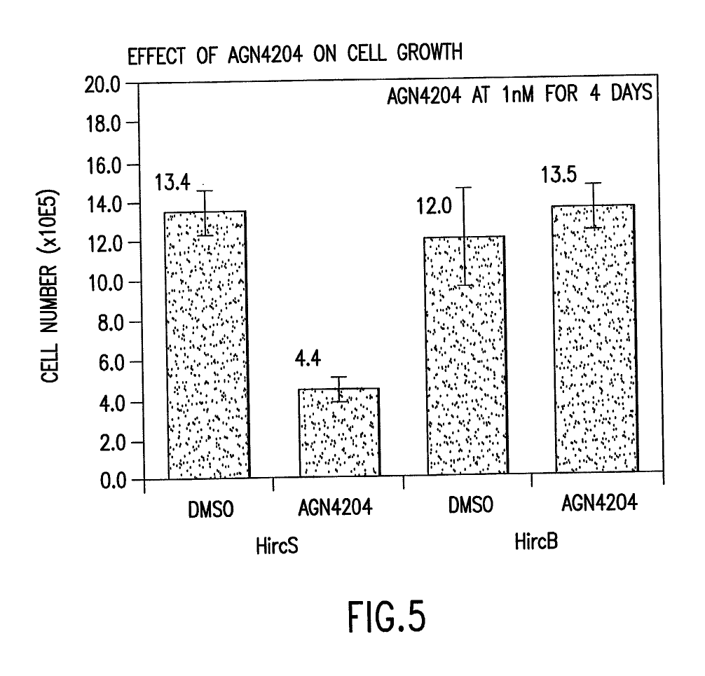

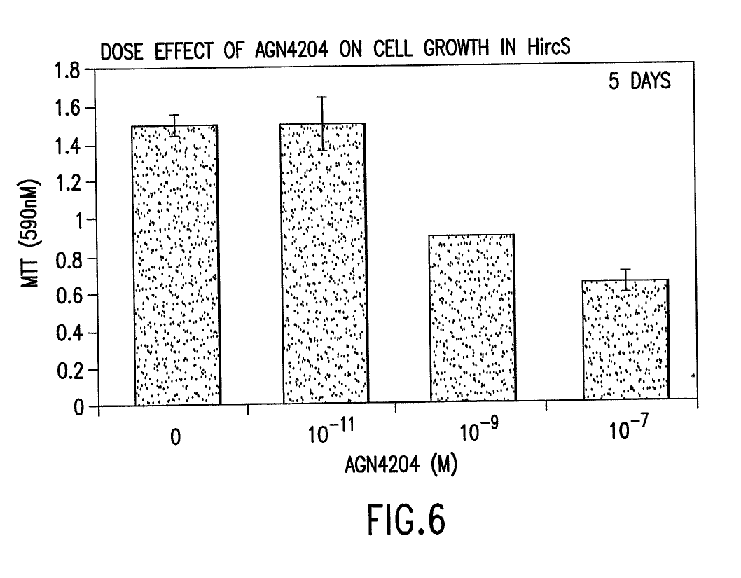

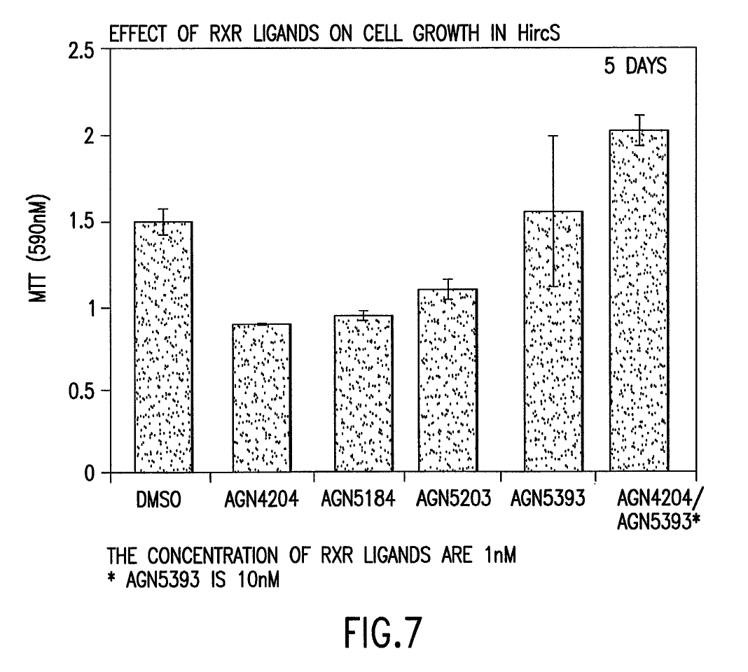

FIGS. 6 and 7 show effects of RXRα ligands on HircRXRα cell growth as determined when cell growth was assessed by the MTT assay. FIG. 6 shows a dose response curve for the effect of AGN 4204 on HircRXRα cells. As indicated, there was a significant reduction in growth of HircRXRα cells in response to five days of treatment with 1 nM AGN 4204 and further reduction in growth in response to 100 nM AGN 4204. FIG. 7 shows the growth after five days of various treatments of HircRXRα cells with agonists and antagonists. As shown, the agonists of Table 1 all decreased the growth of the cell line; the antagonist 5393 had no effect when used alone at 1 nM, but at 10 nM concentrations was able to reverse the effect of AGN 4202.

### Example 4

### Effect of the Presence of RXR Receptors on Response to Insulin

The HircRXRα cells of the invention still can, of course, express the receptor for human insulin. As is understood, the parent cell line HircB responds to contact with insulin; this response, which can be measured in a number of ways, including the insulin stimulated phosphorylation of MEK and of MAP kinase, diminishes after several minutes. The presence of the RXR receptor in the cell appears to sustain the effect of insulin.

To demonstrate this, near confluent cells either of HircB or HircRXRα were incubated overnight in media containing 0.1% fetal calf serum. They were then incubated for various periods in the presence of 100 nM insulin and then lysed. Solubilized cell proteins were subjected to SDS-PAGE and transferred to nitrocellulose and immunoblotted using antibodies specific to phosphorylated forms of MEK and MAP kinase.

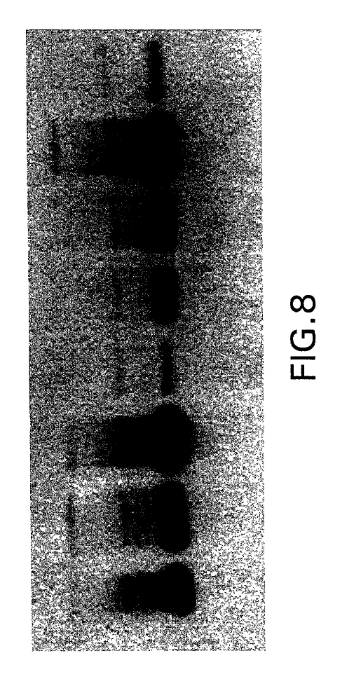

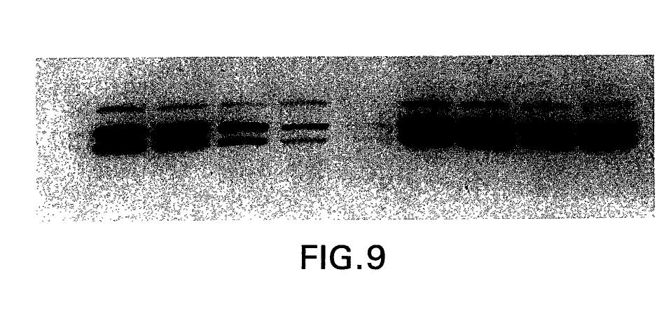

FIGS. 8 and 9 show the results. FIG. 8 shows the effect on phosphorylation of MEK in response to insulin. In the left panel, the parental HircB cell line was tested for phosphorylated MEK in the solubilized protein at 0 minutes (lane 1), 5 minutes (lane 2), 15 minutes (lane 3) and 30 minutes (lane 4) after treatment with 100 nM insulin. As seen, after 5 minutes, a large amount of phosphorylated MEK was present and increased after 15 minutes but this amount had diminished considerably by 30 minutes. On the other hand, in the HircRXRα cells of the invention (lanes 5-8), the initial concentration of insulin-induced phosphorylated MEK is enhanced over the 15 minute interval more significantly, and is less reduced after 30 minutes than in the HircB cell line.

Similar results are shown in FIG. 9 with respect to the phosphorylation of MAP-kinase in response to insulin. In the left panel (labeled 1) the parental cell line shows an initial increase in the phosphorylated forms of MAP-kinase after 5 minutes which is maintained at 15 minutes but diminishes considerably after 45 minutes and is virtually gone after 90 minutes (lanes 1b-1e). In contrast, in HircRXRα cells, shown in the right-hand panel, (labeled 2) the levels of phosphorylated MAP-kinase are substantially maintained over the 90-minute period (lanes 2b-2e).

1. A stable cell line which constitutively and stably expresses both a nucleotide sequence encoding at least one retinoid receptor and a nucleotide sequence encoding an insulin receptor.

2. The cell line of claim 1 which is a transfected rat fibroblast cell line.

3. The cell line of claim 2 which is a transfected HircB cell line.

4. The cell line of claim 1 wherein the retinoid receptor is selected from the group consisting of RXR, RAR, Vitamin D receptor (VDR), thyroid receptor (TR), peroxisome-proliferator activated receptor (PPAR), COUP, and Arp-1.

5. The cell line of claim 4 wherein the retinoid receptor is RXR.

6. The cell line of claim 5 wherein the RXR is RXRα.

7. The cell line of claim 1 which constitutively expresses two nucleotide sequences encoding two different retinoid receptors.

8. The cell line of claim 7 wherein one of said retinoid receptors is RXR.

9. The cell line of claim 8 wherein one of said receptors is selected from the group consisting of RAR, Vitamin D receptor (VDR), thyroid receptor (TR), peroxisome-proliferator activated receptor (PPAR), COUP, and ARP-1.

10. The cell line of claim 9 wherein one of said receptors is PPARγ.

11. The cell line of claim 8 wherein the nucleotide sequence encoding RXR is stably expressed and the nucleotide sequence encoding the other retinoid receptor is transiently expressed.

12. The cell line of claim 7 wherein the nucleotide sequence encoding both retinoid receptors are stably expressed.

13. A method to identify an agonist for a retinoid receptor which method comprises contacting the cell line of claim 1 with a candidate agonist;
observing cell growth in the presence and absence of said candidate agonist;
observing a difference in cell growth in the presence as opposed to the absence of said candidate agonist
whereby a candidate agonist which effects a change in cell growth is identified as an agonist of said retinoid receptor.

14. The method of claim 13 wherein said receptor is RXR.

15. A method to identify an agonist for a retinoid receptor which method comprises contacting the cell line of claim 7 with a candidate agonist;
observing cell growth in the presence and absence of said candidate agonist;
observing a difference in cell growth in the presence as opposed to the absence of said candidate agonist
whereby a candidate agonist which effects a change in cell growth is identified as an agonist of said retinoid receptor.

16. The method of claim 15 wherein one receptor is RXR and the other is selected from the group consisting of RAR, Vitamin D receptor (VDR), thyroid receptor (TR), peroxisome-proliferator activated receptor (PPAR), COUP, and ARP-1.

17. The method of claim 16 wherein said other receptor is PPARγ.

18. A method to identify an antagonist of a retinoid receptor which method comprises contacting the cell line of claim 1 with a candidate compound in the presence of a known agonist of said receptor;
determining any difference in cell growth in the presence and absence of said candidate compound;
whereby a difference in cell growth in the presence of said candidate compound as compared to the absence of said candidate compound identifies said candidate compound as an antagonist.

19. The method of claim 18 wherein said receptor is RXR.

20. A method to identify an antagonist of a retinoid receptor which method comprises contacting the cell line of claim 7 with a candidate compound in the presence of a known agonist of said receptor;
determining any difference in cell growth in the presence and absence of said candidate compound;
whereby a difference in cell growth in the presence of said candidate compound as compared to the absence of said candidate compound identifies said candidate compound as an antagonist.

21. The method of claim 20 wherein one receptor is RXR and the other is selected from the group consisting of RAR, Vitamin D receptor (VDR), thyroid receptor (TR), peroxisome-proliferator activated receptor (PPAR), COUP, and ARP-1.

22. The method of claim 21 wherein said other receptor is PPARγ.
